(19) United States
(12) Patent Application Publication
(10) Pub. No.: US 2006/0235382 A1
(43) Pub. Date: Oct. 19, 2006

(54) Instrument guard and reusable medical device employing same

(76) Inventors: Yoram Cohen, Shoham (IL); Abraham Mates, Givat Shemuel (IL); Gadi Porat, Jerusalem (IL); Ilan Yotvat, Rosh HaAyin (IL)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/109,052

(22) Filed: Apr. 19, 2005

(51) Int. Cl. A61B 18/18

(52) U.S. Cl. 606/51

(57) ABSTRACT

An instrument guard for a MIS medical device includes an attachment portion and an instrument holding portion. The attachment portion is removably attachable to a containment tube of a medical device used for minimally invasive surgery. The instrument holding portion is used to contain and insulate surgical instruments used for the procedure. The instrument guard may be removed after use, exposing the surgical instruments and allowing for cleaning thereof, by washing, brushing, ultrasonic cleaning and visual inspection. A clean instrument guard is then placed on the device, enabling the device to be reused in another procedure.

### FIELD OF THE INVENTION

The present invention relates to medical devices and, more particularly, to devices which can be used in minimally invasive surgery.

### BACKGROUND OF THE INVENTION

Minimally invasive surgery (MIS) is a term for invasive procedures using instruments that help to minimize the size of the incisions. A typical instrument used in MIS is a laparoscope or an endoscope. A laparoscope is a narrow tube which is placed in the surgical site, and typically includes a surgical instrument (such as a knife or cutting instrument) and a forceps for holding tissue at its distal end. Often, the cutting instrument and/or forceps are electro-surgical and sometimes laser based. Electro-surgical devices can be monopolar, wherein only one electrode is positioned on the device and the other one is placed on the patient, causing the energy to pass through the patient's body. Alternatively, electro-surgical devices can be bipolar, wherein both electrodes are located on the device, and the energy is contained at the surgical site.

An example of a bipolar device for MIS is disclosed in U.S. Pat. No. 5,445,638 to Rydell et al. The device disclosed therein is a bipolar electro-surgical device having an elongated tubular member with bipolar tissue cutting forceps and a cutting instrument. Two conductor pairs extend in parallel arrangement on the forceps, and the cutting instrument extends between the forceps.

Devices such as the one disclosed in U.S. Pat. No. 5,445,638 are generally expensive, and as such, it would be useful to have such a device which can be reused safely. A particular difficulty in providing a reusable device is that there is a high probability of tiny fragments or portions of tissue being left on the inner parts of the instrument, particularly the cutting instrument which is positioned between the forceps. This can lead to complications during subsequent procedures using the same device. As the cutting instruments are contained within the device, they are generally inaccessible and very difficult to clean.

A prior art device disclosed in U.S. Pat. No. 6,840,932 to Lang seeks to address this problem by providing a device with a flushing connection piece, allowing an air or liquid to be introduced into the shaft of the device, flushing and cleaning out the channel. However, without direct access to the inner components of the device, it is difficult to ascertain that sufficient cleaning has been accomplished.

A reusable endoscope is disclosed in U.S. Pat. No. 5,928,255 to Meade et al. The instrument includes a tool assembly and a detachable handle. The entire tool assembly consisting of a sleeve, and extension and a jaw assembly are removable as a single unit from the handle assembly, facilitating the cleaning process. However, in the embodiments described therein, cleaning is also done via flushing of a cleaning solution, without direct access to the surgical instruments.

There is thus a widely recognized need for, and it would be highly advantageous to have, a device which is suitable for MIS, which can be readily cleaned and reused.

### SUMMARY OF THE INVENTION

The present invention thus aims to provide an instrument guard, which can be used to hold surgical instruments in place and can be removed to expose the instruments for cleaning.

According to one aspect of the invention, there is provided an instrument guard for use with a medical device having an instrument portion having at least one surgical instrument including an instrument proximal end and an instrument distal end and a shaft portion therebetween and a containment tube partially surrounding the instrument portion and partially leaving an exposed portion of the instrument portion. The instrument guard includes a flexible, insulating, tubular member including an attachment portion at a proximal end thereof for removable attachment to the containment tube and an instrument holding portion at a distal portion thereof for removable holding of the exposed portion of the instrument portion, and an inner diameter reducing portion incorporated in the instrument holding portion.

According to another aspect of the invention, there is provided a medical device for insertion into a body, which includes an instrument portion having at least one surgical instrument with an instrument proximal end, an instrument distal end, and a shaft therebetween, and a containment tube partially surrounding the shaft and partially leaving an exposed portion of the shaft, and an instrument guard removably positionable on the exposed portion of the shaft.

According to yet another aspect of the invention, there is provided a method for enabling reuse of a medical device for an invasive procedure. The method includes providing a medical device having at least one surgical instrument with a proximal end, and distal end, and a shaft therebetween, a containment tube partially surrounding the shaft, an instrument guard partially surrounding the shaft, the instrument guard having a distal end which is distal to a distal end of the containment tube and is proximal to a distal end of the surgical instrument, inserting the medical device in a body, removing the medical device from the body, removing the instrument guard so as to expose a distal portion of the shaft, cleaning the exposed distal portion of the shaft, placing a new instrument guard around at least a portion of the exposed distal portion to replace the removed instrument guard, and sterilizing the medical device.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a partially cut, schematic side view of a PRIOR ART medical device;

FIG. 2A is a partially cut, schematic side view of a medical device including an instrument guard, constructed and operative in accordance with a preferred embodiment of the present invention;

FIG. 2B is an enlarged view of a bipolar forceps configuration, in accordance with the embodiment depicted in FIG. 2A;

FIG. 2C is an enlarged, cross-sectional view of a portion of the medical device of FIG. 2C, depicting the instrument guard in place on the device;

FIG. 3A is a schematic, cut-away illustration of an instrument guard constructed in accordance with a preferred embodiment of the present invention;

FIG. 3B is a perspective view of instruments held in a containment tube showing an exposed portion of the shaft;

FIG. 3C is a perspective view of the instruments of FIG. 3B, depicting the instruments separated from one another so as to facilitate cleaning;

FIGS. 4A and 4B are a schematic illustration and a close-up, cross-sectional view, respectively, of a device with a stop, in accordance with one embodiment of the present invention;

FIGS. 5A and 5B are cross-sectional illustrations of an instrument guard, in accordance with embodiments of the present invention; and

FIGS. 6A, 6B and 6C are illustrations of a surgical instrument included in the device of the present invention having attachment points at a proximal end of the shaft, in the middle of the shaft, and on the instrument itself, respectively.

### DESCRIPTION OF THE PREFERRED EMBODIMENTS

The present invention is an instrument guard, for removably holding surgical instruments for minimally invasive surgery (MIS). The instrument guard is used in combination with a modified device for MIS, such as a laparoscope or endoscope, and provides access to surgical instruments following a procedure, allowing them to be cleaned and reused.

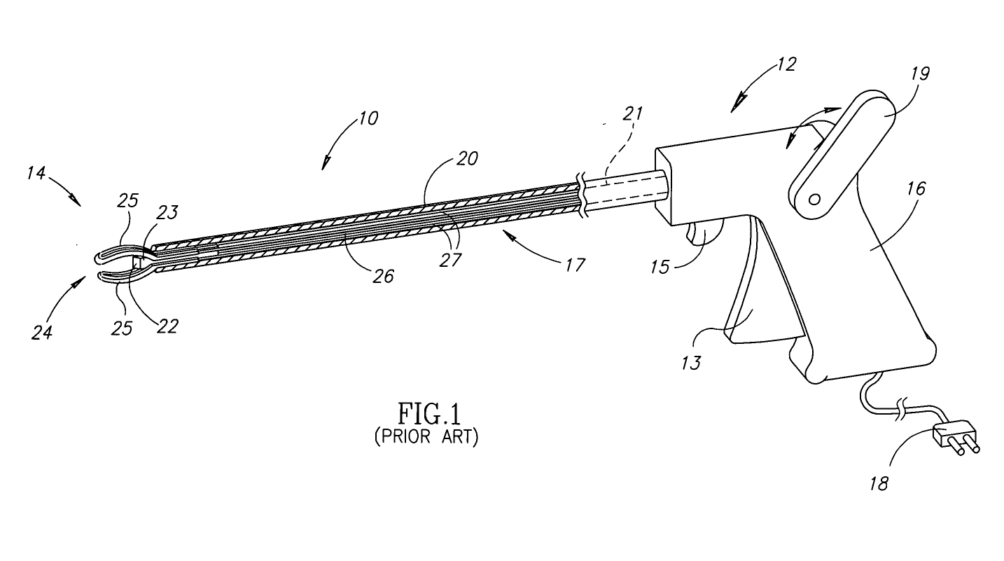

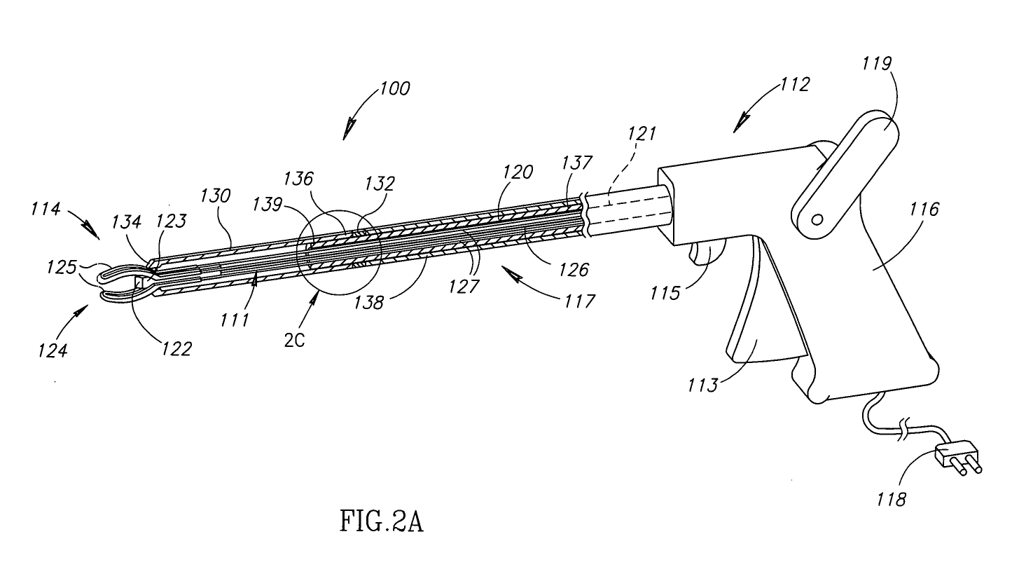

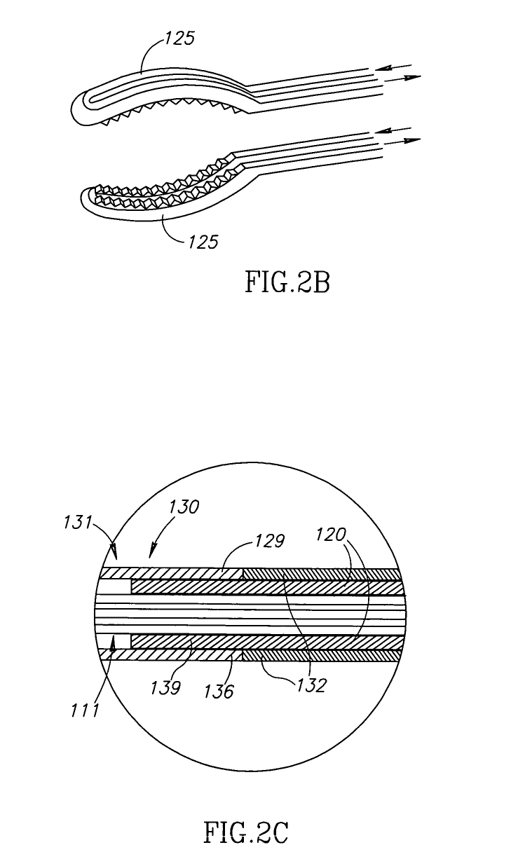

For purposes of better understanding the present invention, as illustrated in FIGS. 2-6 of the drawings, reference is first made to the construction and operation of a conventional (i.e., prior art) laparoscope as illustrated in FIG. 1.

FIG. 1 is a partially cut, schematic side view of a prior art medical device 10 used for minimally invasive surgery. Device 10 has a proximal end 12 and a distal end 14. Proximal end 12 includes a handle portion 16. An instrument portion 17 extends distally from handle portion 16. Instrument portion 17 includes at least one instrument 22 used in performing a MIS procedure. In one embodiment, instrument 22 is a blade, scissors, or other cutting instrument. In another embodiment, instrument 22 is an electro-surgical instrument. In another embodiment, instrument 22 is a laser for performing laser surgery. It should be readily apparent that several such instruments 22 can be used in combination and that for the purpose of the present application, the term instrument 22 defines one or more instruments used in MIS. Instrument 22 has a shaft 26 connecting an instrument proximal end 21 and an instrument distal end 23. Instrument proximal end 21 is operatively connected to handle portion 16, and specifically to an instrument control 19 included within handle portion 16. Instrument control 19 of handle portion 16 is configured to control movements of instrument 22. Specifically, by pushing instrument control 19 in a forward (distal) motion, instrument 22 is pushed forward in the direction of the surgical site. Release of instrument control 19 causes instrument 22 to move back into its default (withdrawn) position. Instrument portion 17 further includes a forceps 24 for grasping tissue, having a shaft portion 27 which extends proximally from forceps 24 to handle portion 16 and jaws 25 for grasping tissue. For a bipolar device, each jaw 25 of forceps 24 acts as an electrode. A forceps control 13 is operatively connected to both forceps 24 and handle portion 16, and allows the surgeon to control opening and closing of forceps 24. In the case of an electro-surgical device, a current control 15 is also operatively connected to handle portion 16, and is used for control of current flow into the electro-surgical component of instrument portion 17, which in the present embodiment is bipolar forceps 25. Wires connecting current control 15 to the electro-surgical component run alongside shaft 26. The device is powered by an external power source, attached via a power cord 18. Thus, a user can manually and/or electronically control each of the instruments 22 and forceps 24 via various controls operatively connected to handle portion 16.

An extended containment tube 20 surrounds shaft 26 and any additional wires and tubes running along a length of the device 10. Typically, extended containment tube 20 extends from handle portion 16 to distal end 14. In one embodiment, forceps 24 and instrument 22 are both included in device 10, as shown in FIG. 1. When device 10 is not in use, jaws 25 of forceps 24 extend distally past the extended containment tube 20, and instrument 22 is substantially contained within extended containment tube 20, as shown in FIG. 1. During use, forceps control 13 can be used to both close jaws 25, placing them in contact with one another, and to push extended containment tube 20 forward (distally), causing at least a portion of jaws 25 to be contained within extended containment tube 20. Furthermore, during use instrument control 19 can be used to push instrument 22 forward, causing it to exit extended containment tube 20. In an alternative embodiment, instrument 22 is a scissor, and forceps 24 may or may not be present. However, it should be noted that in all possible configurations, extended containment tube 20 encloses the entire shaft 26 of instrument 22 and also shaft portion 27 of forceps 24, when present.

Devices such as the prior art device 10 described above are generally not suitable for reuse, since it is extremely difficult to properly clean the instruments within extended containment tube 20. Even devices which provide means for cleaning, such as a flushing system, are not sufficiently rigorous and may still allow for small pieces to be left inside. The present invention addresses this problem, by providing a device whose instruments are readily accessible for cleaning, by washing, brushing and visual inspection, as described in further detail hereinbelow.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Reference is now made to FIG. 2A, which is a partially cut, schematic side view of a medical device 100 constructed and operative in accordance with a preferred embodiment of the present invention. Similar to the prior art device 10 depicted in FIG. 1, device 100 has a proximal end 112 and a distal end 114. Proximal end 112 includes a handle portion 116. An instrument portion 117 extends distally from handle portion 116. Instrument portion 117 includes at least one instrument 122 used in performing a MIS procedure. In one embodiment, instrument 122 is a blade, scissors, or other cutting instrument. In another embodiment, instrument 122 is an electro-surgical instrument. In another embodiment, instrument 122 is a laser for performing laser surgery. It should be readily apparent that several such instruments 122 can be used in combination and that for the purpose of the present application, the term instrument 122 defines one or more instruments used in MIS. Instrument 122 has a shaft 126 connecting an instrument proximal end 121 and an instrument distal end 123. Instrument proximal end 121 is operatively connected to handle portion 116, and specifically to an instrument control 119 included within handle portion 116. Instrument control 119 of handle portion 116 is configured to control movements of the instrument 122. Specifically, by pushing instrument control 119 in a forward (distal) motion, instrument 122 is pushed forward in the direction of the surgical site. Release of instrument control 119 causes instrument 122 to move back into its default (withdrawn) position. In a preferred embodiment, instrument portion 117 further includes a forceps 124 for grasping tissue, having a shaft portion 127 which extends proximally from forceps 124 to handle portion 116. For a bipolar device, each jaw 125 of forceps 124 acts as an electrode, as shown in FIG. 2B. A forceps control 113 is operatively connected to both forceps 124 and handle portion 116, and allows the surgeon to control opening and closing of forceps 124. In the case of an electro-surgical device, a current control 115 is also operatively connected to handle portion 116, and is used for control of current flow into the electro-surgical component of instrument portion 117, which in the present embodiment is bipolar forceps 125. Wires connecting current control 115 to the electro-surgical component run alongside shaft 126. The device is powered by an external power source attached via a power cord 118. Thus, a user can manually and/or electronically control each of the instruments 122 and forceps 124 via various controls operatively connected to handle portion 116.

A containment tube 120 surrounds shaft 126 and any additional shafts, wires or tubes running along a length of the device 100. Containment tube 120 has a proximal end 137 and a distal end 139. However, containment tube 120 is shorter than extended containment tube 20 of the prior art device described with reference to FIG. 1. That is, containment tube 120 surrounds only a portion of shaft 126 and shaft portion 127, exposing a distal portion of shaft 126 and shaft portion 127. These distal portions are removably covered by an instrument guard 130, as described further hereinbelow. Thus, exposed portions 111 of shaft 126 and shaft portion 127 are free to move with respect to one another when contained within containment tube 120 without instrument guard 130. Exposed portion 111 can be any suitable length, but should be an amount which allows relative movement between shaft 126 (of one or several instruments 122) and shaft portion 127. In a preferred embodiment, containment tube 120 is comprised of a biocompatible metal, but it should be readily apparent that any strong biocompatible material can be used. Preferably, containment tube 120 should have heat and electrical insulating properties as well.

During a procedure, instrument guard 130 having a distal end 134 and a proximal end 136 is positioned on exposed portion 111 of shaft 126 and shaft portion 127. The instrument guard 130 contains instruments 122 and forceps 124 for control of movement, while also insulating the body from heat and electrical conductance. A closer view of instrument guard 130 positioned on device 100 is shown in FIG. 2C in cross-section. Instrument guard 130 is a tubular member having an attachment portion 129 and an instrument holding portion 131. Attachment portion 129 is configured for attachment to a distal portion of containment tube 120. Instrument holding portion 131 contains exposed portion 111 of shaft 126 and shaft portion 127, thus replacing a distal portion of extended containment tube 20. In a preferred embodiment, instrument guard 130 is comprised of a flexible material, most preferably silicon. For the purposes of the present application, the term “flexible” is defined to include flexible and semi-flexible materials. The material of instrument guard 130 is preferably flexible enough to be removably positionable around containment tube 120, and may adhere to containment tube 120 by friction between the materials of attachment portion 129 and containment tube 120. In alternative embodiments, attachment portion 129 of instrument guard 130 is mechanically or chemically attachable to containment tube 120. For example, attachment portion 129 may include mechanical attachment means such as a clip, screw, or any other suitable attachment mechanism. Alternatively, attachment portion 129 may include a sealant, which can be used to removably attach attachment portion 129 to containment tube 120. In a preferred embodiment, the material of instrument guard 130 is a material which can insulate from heat and electricity during electro-surgical procedures. Furthermore, the material of instrument guard 130 is a material which is resistant to deformation in the presence of heat. In a preferred embodiment, attachment portion 129 and instrument holding portion 131 are comprised of a single, continuous material. In alternative embodiments, different materials can be used for attachment portion 129 and instrument holding portion 131. In one embodiment, the material of instrument guard is shaped to surround the contours of instruments 122 held within said instrument holding portion. Instrument guard 130 can be disposable or reusable.

In order to ensure easy and accurate mounting of the instrument guard 130 onto the distal end 139 of containment tube 120, there is preferably also provided a stop 132. Stop 132 may be attached by glue, pressure, or any other suitable attachment means to containment tube 120, at an area just proximal to where proximal end 136 of instrument guard 130 lies when in place on device 100. Stop 132 stops proximal end 136 of instrument guard 130 from being advanced proximally over containment tube 120 more than is necessary, and has an outer diameter which is less than or equal to the outer diameter of instrument guard 130. In one embodiment, stop 132 is a ring, as shown in FIG. 2A. In this embodiment, a secondary tube 138 may surround containment tube 120, extending proximally from a proximal side of stop 132 to a proximal end of containment tube 120. Secondary tube 138 may be removable or non-removable, and serves to keep an outer diameter of instrument portion 117 relatively constant. Alternatively, if stop 132 is a ring and no secondary tube 138 is present, stop 132 may have a tapered proximal portion, such that the transition from stop 132 to containment tube 120 is smooth. In a preferred embodiment, stop 132 is a tube which extends from proximal end 136 of instrument guard 130 to a proximal end of containment tube 120, as shown in FIG. 2C. In this embodiment, stop 132 serves to keep an outer diameter of instrument portion 117 relatively constant. Stop 132 may be removable or non-removable and further may be reusable or disposable. Stop 132 is comprised of any suitable, biocompatible material, such as silicon, plastic or metal. Stop 132 may further be comprised of insulating material, potentially eliminating the need for containment tube 120 to be comprised of insulating material.

In one embodiment, stop 132 has a slit along its length, enabling it to positioned on device 100 from the side rather than being slid on from the distal end of device 100. This configuration provides ease of positioning and removing of stopper 132.

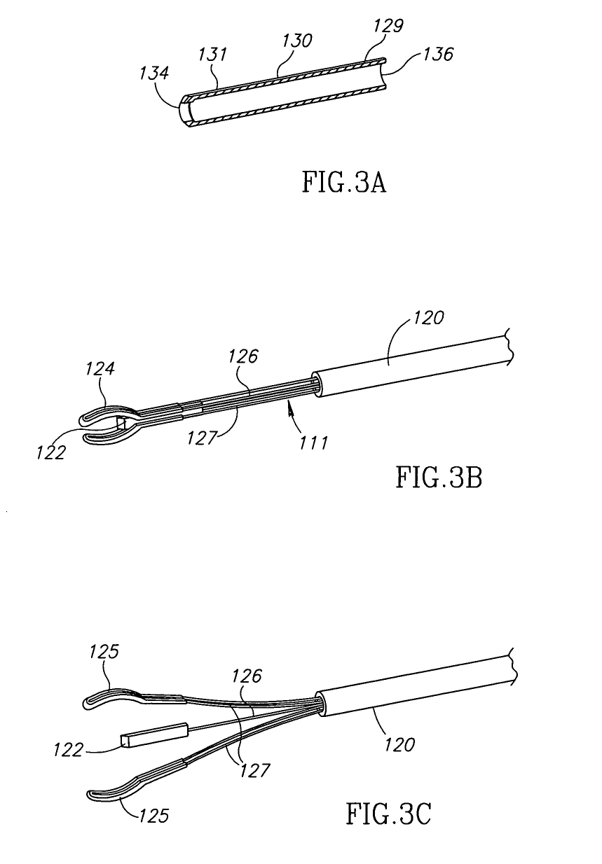

Referring now to FIGS. 3A-C, instrument guard 130 is depicted in FIG. 3A, instruments in containment tube 120 showing exposed portion 111 are depicted in FIG. 3B, and instruments in containment tube 120 in a separated position are depicted in FIG. 3C. As shown in FIG. 3A, instrument guard 130 has a distal end 134 and a proximal end 136, and includes attachment portion 129 and instrument holding portion 131. As shown in FIG. 3B, shaft 126 and shaft portion 127 are partially contained within containment tube 120. Exposed portion 111 of shaft 126 and shaft portion 127 is accessible with instrument guard 130 removed. As shown in FIG. 3C, instruments 122 and forceps 124 are separatable from one another when instrument guard 130 is removed. This allows for cleaning, by washing, brushing and visual inspection of each of the instruments 122 and/or forceps 124. Cleaning may also be done by ultrasonic methods commonly known in the art.

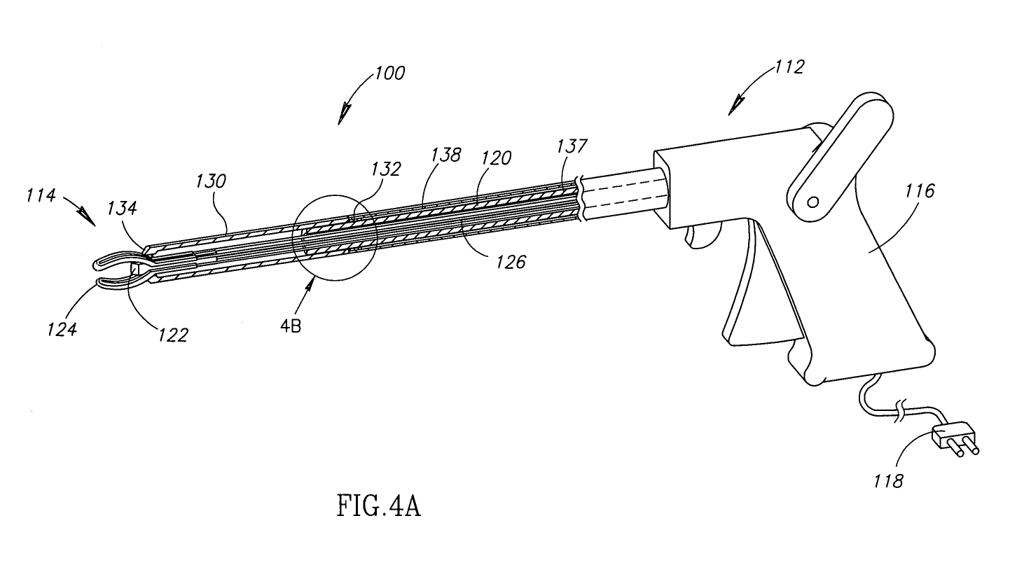

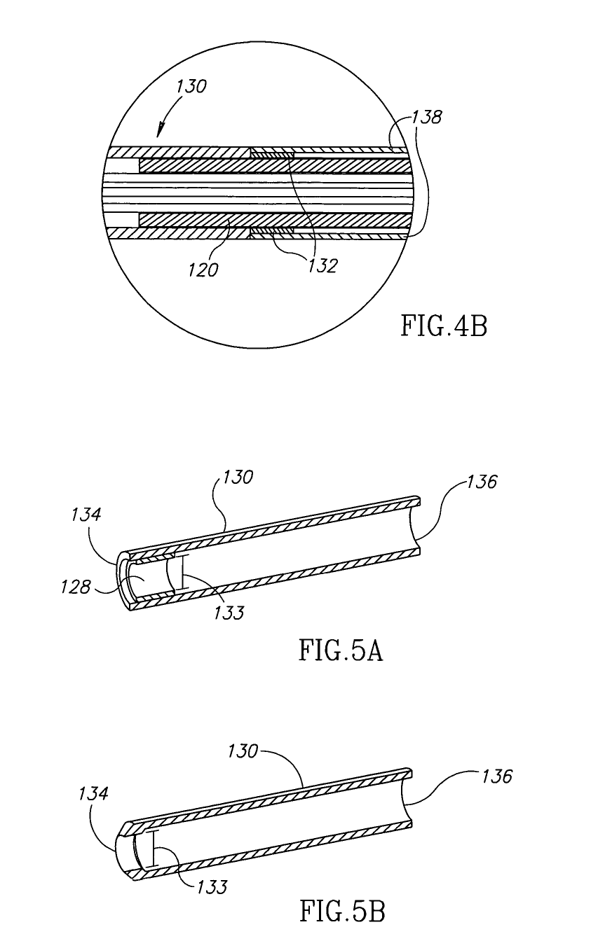

Reference is now made to FIGS. 4A and 4B, which are a schematic illustration and a close up, cross-sectional view, respectively, of a device 100 with a stop 132 in accordance with another embodiment of the present invention. Stop 132 is a ring having a smaller outer diameter than an outer diameter of instrument guard 130. A secondary tube 138 having a distal end and a proximal end is included, wherein the distal end of secondary tube 138 covers stop 132, and the proximal end of secondary tube 138 is positioned at the proximal end 137 of containment tube 120. Secondary tube 138 keeps the outer diameter of instrument portion 117 constant. An additional use of secondary tube 138 is to further insulate the instruments and/or help keep them clean. Secondary tube 138 can be comprised of the same material as instrument guard 130 or stop 132, or may be comprised of different material.

In one embodiment, secondary tube 138 has a slit along its length, enabling it to positioned on device 100 from the side rather than being slid on from the distal end of device 100. This configuration provides ease of positioning and removing of secondary tube 138.

In another embodiment, instrument guard 130 is configured to extend proximally to handle portion 116. In this embodiment, stop 132 and/or secondary tube 138 are not included.

In order for instrument guard 130 to fit over containment tube 120, an inner diameter of instrument guard 130 must be slightly greater than an inner diameter of containment tube 120. However, it is preferable that at distal end 114 of device 100, the inner diameter approximates the inner diameter of containment tube 120, so that the instruments contained therein are not subject to loose and unnecessary movement. As such, possible designs for distal end 134 of instrument guard 130 are depicted in FIGS. 5A and 5B. In one embodiment, shown in FIG. 5A, an inner diameter reducing portion 128 is placed at distal end 134. Inner diameter reducing portion 128 is simply a small tube or ring having an inner diameter 133 approximately equal to the inner diameter of containment tube 120. Inner diameter reducing portion 128 is either intrinsic to or attachable to an inner wall of instrument guard 130. In another embodiment, as shown in FIG. 5B, distal end 134 is tapered, such that distal end 134 itself has an inner diameter 133 which approximates the inner diameter of containment device 120.

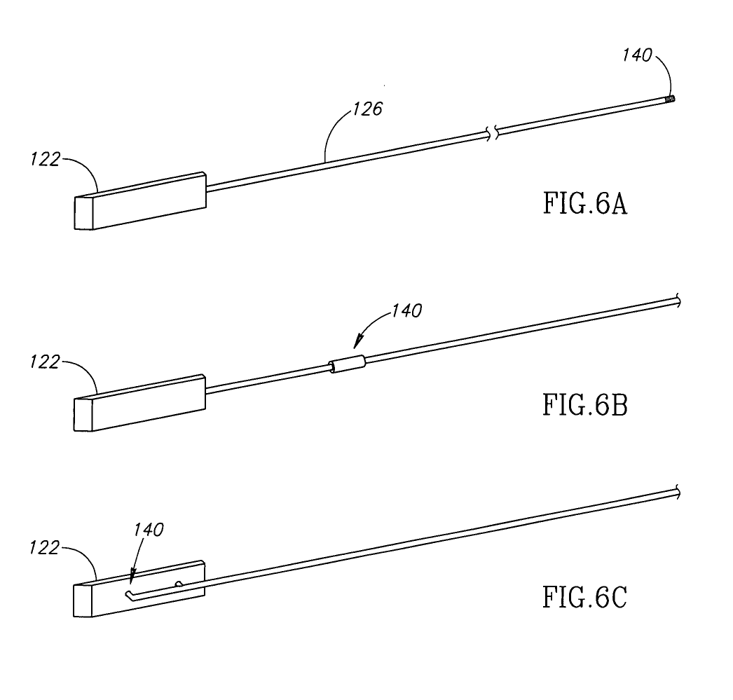

Reference is now made to FIGS. 6A-6C, which are illustrations of an instrument 122 which is removable from device 100 in accordance with several embodiments. In a first embodiment, shown in FIG. 6A, a proximal end of shaft 126 has connector a 140, which can be used to connect and disconnect blade 122 or other surgical tools from handle portion 116 of device 100, further simplifying the process of cleaning or replacement of the tools if necessary. In an alternative embodiment, shown in FIG. 6B, connector 140 is located on shaft 126. In yet another embodiment, a mechanical connector connects blade 122 to shaft 126, allowing for blade 122 to be disconnected from shaft 126. It should be readily apparent that any of the instruments included in device 100 can be configured to separately attach and detach from handle 116 or a shaft 126.

In order to reuse a medical device for an invasive procedure, the device must be cleanable, allowing it to then be re-sterilized. The device of the present invention can be introduced into and removed from a body, generally during a surgical procedure, after which it is cleaned, brushed, visually inspected, partially replaced, and reused. Thorough cleaning, by washing, brushing, ultrasonic cleaning and visual inspection are made possible by removal of instrument guard 130, which exposes the instruments, allowing them to be spread apart. Cleaning is generally done by manual scrubbing and brushing or by ultrasonic cleaning methods, and sterilization is generally accomplished using an autoclave, ethylene oxide, or any other acceptable sterilization method. After cleaning, instrument guard 130 can be easily and inexpensively replaced by a new instrument guard. Stop 132 allows for accurate placement of the new instrument guard. The cleaned device with the new instrument guard is then placed in a suitable package and sterilized by, for example, autoclaving at a temperature of about 120 degrees Celsius, and prepared for insertion into a body for additional use. It is also possible to sterilize the device without the instrument guard in place, and to then place a new, previously sterilized instrument guard, onto device 100 under sterile conditions.

It should be apparent that in the embodiments described above, an outer diameter of device 100 may be larger than the outer diameters of prior art devices such as device 10 depicted in FIG. 1. As such, a larger sized trocar than the current standard sized trocars may be necessary for entry into the surgical site. In a preferred embodiment, a trocar with an appropriately sized diameter is provided. In a preferred embodiment-7 mm. Thus, a kit may be provided, included the device of the present invention, as well as a trocar suitable for use with the device. Alternatively, the thicknesses of containment tube 120, instrument guard 130, stop 132 and secondary tube 138 can all be configured such that the total outer diameter is of a standard size, for use with a standard trocar.

The present invention can be used with bipolar or monopolar electro-surgery devices, non-electro-surgery devices, or any surgical device which can be used for minimally invasive surgery, particularly laparoscopes, endoscopes, and the like.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. For use with a minimally invasive surgery medical device having an instrument portion having at least one surgical instrument including an instrument proximal end and an instrument distal end and a shaft portion therebetween and a containment tube partially surrounding the instrument portion and partially leaving an exposed portion of the instrument portion, an instrument guard comprising:
a. a flexible, insulating, tubular member comprising an attachment portion at a proximal end thereof for removable attachment to the containment tube and an instrument holding portion at a distal portion thereof for removable holding of the exposed portion of the instrument portion; and
b. an inner diameter reducing portion incorporated in said instrument holding portion.

2. The instrument guard of claim 1, wherein said inner diameter reducing portion is a tapered distal tip.

3. The instrument guard of claim 1, wherein said inner diameter reducing portion is an inner ring in contact with an inner wall of said distal portion of said tubular member.

4. The instrument guard of claim 1, shaped to surround contours of instruments held within said instrument holding portion.

5. The instrument guard of claim 1, designed to mimic a distal portion of an extended containment tube which encloses an entire shaft of said instrument portion, wherein said distal portion of said extended containment tube is replaced by said instrument guard.

6. The instrument guard of claim 1, wherein said attachment portion includes a mechanical attachment mechanism for mechanically attaching said instrument guard to said containment tube.

7. The instrument guard of claim 1, wherein said tubular member is resistant to deformation in the presence of heat.

8. The instrument guard of claim 1, wherein said tubular member is an insulating material.

9. The instrument guard of claim 8, wherein said insulating material is a thermally insulating material.

10. The instrument guard of claim 8, wherein said insulating material is an electrically insulating material.

11. The instrument guard of claim 1, wherein said tubular member is formed of silicon.

12. The instrument guard of claim 1, wherein said instrument guard is disposable

13. A medical device for insertion into a body, which comprises:
a. an instrument portion comprising:
at least one surgical instrument having an instrument proximal end, an instrument distal end, a shaft therebetween;
a containment tube partially surrounding said at least one surgical instrument, and partially leaving an exposed portion of said at least one surgical instrument; and
b. an instrument guard removably positionable on said exposed portion of said surgical instrument.

14. The medical device of claim 13, further comprising a handle portion, said handle portion connected to said instrument proximal end and incorporating means for operating said at least one surgical instrument.

15. The medical device of claim 13, wherein said at least one surgical instrument is an electro-surgical instrument.

16. The medical device of claim 15, wherein said electro-surgical instrument is monopolar.

17. The medical device of claim 15, wherein said electro-surgical instrument is bipolar.

18. The medical device of claim 13, wherein said at least one surgical instrument is a cutting instrument.

19. The medical device of claim 13, further comprising a stop on said containment tube for facilitating accurate mounting of said instrument guard onto a distal end of said containment tube.

20. The medical device of claim 19, wherein said stop is configured to prevent said instrument guard from being pushed proximally on said containment tube past a point at which said instrument guard would not be accurately positioned on said exposed portion.

21. The medical device of claim 19, wherein said stop is a ring having an outer diameter which is greater than an inner diameter of said instrument guard.

22. The medical device of claim 19, wherein said stop is a sleeve having an outer diameter which is greater than an inner diameter of said instrument guard.

23. The medical device of claim 19, wherein an outer diameter of said stop is equal to an outer diameter of said instrument guard.

24. The medical device of claim 19, further comprising a secondary tube extending proximally from said stop to said instrument proximal end.

25. The medical device of claim 24, wherein an outer diameter of said stop is smaller than an outer diameter of said instrument guard, and wherein a distal portion of said secondary tube lies of top of said stop, said distal portion of said secondary tube being thinner than a proximal portion of said secondary tube.

26. The medical device of claim 14, wherein said containment tube extends distally to said handle portion.

27. The medical device of claim 13, wherein said instrument guard comprises:
a. a flexible, insulating, tubular member comprising an attachment portion at a proximal end thereof and an instrument holding portion at a distal portion thereof; and
b. an inner diameter reducing portion incorporated in said instrument holding portion.

28. The medical device of claim 13, wherein said instrument guard is disposable.

29. The medical device of claim 27, wherein said inner diameter reducing portion is a tapered distal tip.

30. The medical device of claim 27, wherein said inner diameter reducing portion is an inner ring in contact with an inner wall of said distal portion of said guard.

31. The medical device of claim 27, wherein said attachment portion lies over a distal portion of said containment tube.

32. The medical device of claim 27, wherein said attachment portion includes a mechanical attachment mechanism for mechanically attaching said instrument guard to said containment tube.

33. The medical device of claim 27, wherein said tubular member is resistant to deformation in the presence of heat.

34. The medical device of claim 27, wherein said tubular member is an insulating material.

35. The medical device of claim 34, wherein said insulating material is a thermally insulating material.

36. The medical device of claim 34, wherein said insulating material is an electrically insulating material.

37. The medical device of claim 27, wherein said tubular member is formed of silicon.

38. The medical device of claim 14, wherein said at least one surgical instrument is detachable from said handle portion.

39. The medical device of claim 38, wherein said at least one surgical instrument is detachable at a location selected from the group consisting of: a distal end of the shaft of the surgical instrument, a proximal end of the shaft of the surgical instrument, and a position along the shaft of the surgical instrument.

40. A method for enabling reuse of a medical device for an invasive procedure, the method comprising:
providing a medical device, the device including one or more surgical instruments having a proximal end, a distal end and a shaft therebetween, a containment tube partially surrounding the shaft and partially exposing the shaft thereby providing an exposed portion of surgical instruments, and an instrument guard removably surrounding the exposed portion of the surgical instruments;
inserting said medical device into the body of a subject;
removing said medical device from the body;
removing said instrument guard so as to expose said exposed portion of said at least one surgical instrument;
cleaning said exposed portion of said at least one surgical instrument;
placing a new instrument guard around at least a portion of said exposed distal portion to replace said removed instrument guard; and
sterilizing said medical device.

41. The method of claim 40, wherein said cleaning includes brushing.

42. The method of claim 40, further comprising visually inspecting said exposed portion prior to said placing a new instrument guard.

43. The method of claim 40, wherein said inserting and removing said medical device are steps in a minimally invasive surgery procedure.

44. For use with a medical device having an instrument portion having at least one surgical instrument including an instrument proximal end and an instrument distal end and a shaft portion therebetween and a containment tube partially surrounding the instrument portion and partially leaving an exposed portion of the instrument portion, and a flexible, insulating instrument guard for removable holding of the exposed portion, wherein the medical device has a specific total outer diameter,
a trocar having a diameter which is suitable for forming an access hole for said medical device.

45. The trocar of claim 44, wherein said diameter is within a range of 6-13 millimeters.
